(19) United States
(12) Patent Application Publication
(10) Pub. No.: US 2005/0085745 A1
(43) Pub. Date: Apr. 21, 2005

(54) Hollow needle holder

(75) Inventors: Johann Karl Kitta, Waldbrol-Herfen (DE); Helmut Bruchmann, Wiehl (DE)

Correspondence Address: WILLIAM COLLARD;COLLARD & ROE, P.C., 1077 NORTHERN BOULEVARD, ROSLYN, NY, 11576, US

(73) Assignee: Johann Karl Kitta

(21) Appl. No.: 10/801,287

(22) Filed: Mar. 16, 2004

(30) Foreign Application Priority Data
Oct. 20, 2003 (DE) 103 48 603.8
Nov. 18, 2003 (DE) 203 17 798.3
Dec. 3, 2003 (DE) 203 18 879.9

(51) Int. Cl. A61M 5/00

(52) U.S. Cl. 600/576; 604/110

(57) ABSTRACT

A hollow needle holder includes a hollow needle container, a hollow needle fixing device, and an unlocking mechanism. The hollow needle container has a first face including a closable opening and a second face. The hollow needle fixing device is disposed on the second face for receipt of a hollow needle or an adapter for a hollow needle projecting through the hollow needle fixing device into the hollow needle container. The unlocking mechanism releases the hollow needle or adapter after use so that the hollow needle or adapter falls into the hollow needle container after activation of the unlocking mechanism.

### CROSS REFERENCE TO RELATED APPLICATIONS

Applicants claim priority under 35 U.S.C. §119 of German Application No. 103 48 603.8 filed Oct. 20, 2003, German Utility Model Application No. 203 17 798.3 filed Nov. 18, 2003 and German Utility Model Application No. 203 18 879.9 filed Dec. 3, 2003.

### BACKGROUND OF THE INVENTION

1. Field of the Invention

The present invention relates to a hollow needle holder, which permits disposal of the hollow needle by means of an elongated hollow needle container. The container has an opening that can be closed on a first face, and a hollow needle fixing device is arranged on the opposite, second face, for accommodating a hollow needle that projects through the hollow needle fixing device into the hollow needle container. An unlocking mechanism releases the hollow needle after use. The hollow needle fixing device can also accommodate, in place of the hollow needle, an adapter which projects through the hollow needle fixing device into the hollow needle container and which can be released by means of the unlocking mechanism after use. The adapter can, for example, be set onto a hollow needle that is already located in the vein of the patient.

2. The Prior Art

Hollow needle holders are used in the medical care of patients, particularly when taking venous blood samples. Together with a hollow needle and a blood sample tube, they are part of a blood sampling set, which usually consists of three pieces.

When a blood sample is taken, a fresh hollow needle is usually first attached to the hollow needle holder. For this purpose, most commercially available hollow needles have an adapter having an outside thread, affixed on the hollow needle, which is screwed into the hollow needle holder. Afterwards, the end of the hollow needle that projects out of the holder is introduced into a vein of the patient. A blood sample tube is set onto the end of the hollow needle that is located in the holder. This tube can be replaced with new tubes, depending on the amount of blood required or the studies to be conducted. After the blood has been taken, the hollow needle is removed from the vein and disposed of. However, during disposal there is the risk that the personnel will accidentally suffer a needle stick wound. Such an injury can have serious consequences, since the patient's blood contained in the hollow needle can be the carrier of infectious diseases.

In order to reduce the risk of such injuries, it is proposed, for example, in EP 0 315 306 A1, for a syringe, to insert the hollow needle set onto the syringe into a container after use, and to close the container. However, since the container has to be held in place during insertion, there is a particularly great risk of injury in this connection.

This problem is avoided by the syringe described in WO 93/18808, in which the hollow needle is retracted into a front chamber after use, which chamber is separated from the actual syringe chamber by means of a diaphragm. In order to prevent re-use, an automatic retraction system can be triggered by deeply pressing the piston after use, thereby retracting the hollow needle into the front chamber. However, this retraction cannot be done immediately after a blood sample is taken, since the blood first has to be removed from the syringe chamber before the piston can be pressed deeply again. This removal typically does not happen until later, in the laboratory, so that there is still a significant risk of injury until then. In addition, painful injuries to the patient can occur if the piston is accidentally pressed in too far while the blood sample is being taken. Also, multiple use of the hollow needle by means of setting on several blood sample tubes is no longer possible.

EP 0 943 352 A2 describes a hollow needle holder in which the hollow needle can be ejected from the holder after use by means of a trigger mechanism. For this purpose, the user positions the hollow needle holder with the contaminated hollow needle above a corresponding disposal container and activates a slide, thereby causing the hollow needle to be released from the holder and to drop into the container. However, there is the disadvantage that the hollow needle can accidentally fall next to the container, as well, and then has to be disposed of by hand. This possibility can easily result in needle stick injuries. The same holds true if the ejection mechanism is triggered at an undesirable point in time. Finally, during ejection of the hollow needle, blood from the hollow needle may also get onto the holder, which can actually be re-used. If the holder is then re-used, there is an additional risk of infection that proceeds from the holder. In order to prevent this possibility, complicated disinfection of the holder is necessary.

### SUMMARY OF THE INVENTION

It is therefore an object of the present invention to provide a hollow needle holder for which disposal of the hollow needle after use takes place in a particularly safe, quick, and simple manner.

This object is accomplished, according to the invention, by providing a hollow needle holder generally of the type stated initially, but in which the unlocking mechanism is configured so that the hollow needle and/or the adapter itself falls into the hollow needle container after activation of the unlocking mechanism.

Before the unlocking mechanism is triggered, the opening formed on the first face of the hollow needle holder, through which the blood sample tubes are set into the one side of the hollow needle or of the adapter, is closed off, for example by means of a plug, a screw-on closure, a cap, or a similar closure means. This arrangement does not present risk of injury, since this closing takes place on the side of the hollow needle holder that faces away from the hollow needle. After the unlocking mechanism is triggered, the hollow needle or the adapter then falls into the hollow needle holder, which is configured to be long enough to completely accommodate the hollow needle and/or the adapter. In this way, any contact with the hollow needle that is contaminated with blood, or with the corresponding adapter and, in particular, any dangerous needle stick injuries are avoided. Before disposal, the second open side of the hollow needle holder, on which the hollow needle and/or the adapter is arranged, can also be closed.

In the following, for simplicity purposes, reference will be made only to the hollow needle, but it is to be understood that the hollow needle can also be replaced by an adapter, in each instance, for example an adapter for setting on a hollow needle.

In order to prevent the hollow needle from falling off the hollow needle holder unintentionally, a locking mechanism is provided, according to the invention, which holds the hollow needle firmly in place on the hollow needle holder. This locking device is configured so that it permits a movement of the hollow needle only in the hollow needle holder. It can be integrated into the unlocking mechanism.

For this purpose, the hollow needle fixing device can be releasably fixed in place on the hollow needle holder by way of an anchoring device. It is particularly advantageous if the hollow needle fixing device is fixed in place on the hollow needle holder from the inside of the holder so that the hollow needle can be screwed in from the outside, for example, through an opening in the hollow needle holder. After the anchoring device is released, the hollow needle fixing device, together with the hollow needle that is screwed in, then falls into the hollow needle holder by itself. In this way, the hollow needle is reliably prevented from falling off, because the hollow needle fixing device is fixed in place on the hollow needle container from the inside, and cannot fall out of the container.

According to a particularly preferred embodiment, the hollow needle fixing device can be engaged on the hollow needle holder. For one thing, the device is reliably fixed in place by means of this engagement. For another, this anchoring can easily be released again by means of forces that act counter to the engagement.

According to the invention, the hollow needle holder can have a passage opening for the hollow needle and at least one attachment opening for the hollow needle fixing device on its second face, whereby the hollow needle fixing device can, in particular, be engaged in the attachment opening. In this connection, the passage openings for the hollow needle and the attachment openings are preferably separate openings. Thus, for example, the hollow needle fixing device can also engage in the passage opening that has been reinforced at the edges, for example, as a result of which the hollow needle fixing device with hollow needle is guided and firmly aligned in a particularly good manner. It is also possible, however, that the passage openings and the attachment openings form a common opening. In a preferred embodiment, a passage opening, particularly a central one, and two attachment openings that lie opposite one another on different sides of the passage opening are provided. This arrangement allows both stable attachment and simple release of the hollow needle fixing device.

The hollow needle fixing device can have at least one flexible arm having a preferably wedge-shaped projection for engagement on the hollow needle fixing device as the means of attachment. In this connection, the arm and the projection are arranged so that the arm passes through the attachment opening in the hollow needle holder from the inside and the projection hooks in on the hollow needle holder from the outside. By exerting pressure on the projection, the projection lifts up from the edge of the attachment opening and the anchoring is released. However, the projection can also serve just for guidance.

For this purpose, the unlocking mechanism preferably has at least one slide, according to the invention, which releases the anchoring device of the hollow needle fixing device on the hollow needle holder. By means of this slide, the force to be exerted to release the anchoring device can be applied in a simple and targeted manner.

This simple and targeted application of force is particularly well possible if the slide has a guide that engages on the anchoring device of the hollow needle fixing device. This guide can be, for example, an arm that narrows at a slant, a groove appropriately formed in the slide, or similar mechanism, whereby a slide can, according to the invention, also have several guides that engage at different anchoring points, for example two arms with grooves that narrow appropriately. In such a case, assembly of the unlocking mechanism is particularly simple, because the unlocking mechanism has only a few individual parts.

Alternatively, according to the invention, two slides can also be provided, each of which preferably engages on a flexible arm.

In order to allow the slide to be operated simply by hand, by means of pressing it from the side, the slide can also project beyond the edge of the hollow needle container. Then it can be activated in a particularly simple manner, for example using the thumb, while the holder is grasped by the hand. By means of this one-hand operation, the risk of injury can be further clearly reduced.

Instead of a slide that projects to the outside, the unlocking mechanism can, according to the invention, also have a cap that is arranged to rotate on the hollow needle container, in which the at least one slide is accommodated so that the slide is moved by rotating the cap. In this connection, it is possible to form the cap and the slide in one piece. Then the cap itself has a guide, so that the anchoring device of the hollow needle fixing device is released during the rotational movement. In this connection, the rotation can take place to the right or to the left, which is advantageous for simple and uncomplicated use.

Alternatively, the cap according to the present invention can have a guide in which a guide element of the slide engages. For two slides, for example, this guide can be a groove in a double-ring shape.

According to the invention, it is particularly advantageous if the unlocking mechanism has a closure device for closing the hollow needle container. This closure device can be formed by a slide, in a particularly simple manner. The slide or slides can also be activated particularly well by means of a rotating cap, whereby the closure effect is preferably achieved both by a left rotation and by a right rotation. In this way, it is possible to reliably achieve the result that the hollow needle holder that serves as the disposal container is always closed, without the second face having to be sealed with an additional manipulation, for example by applying a cap.

According to another aspect of the present invention, the handling of the hollow needle fixing device during the procedure of taking blood is also improved. For this purpose, according to the invention, a hollow needle holder having an opening on a first face, particularly a closable opening, is provided with a hollow needle fixing device for accommodating a hollow needle and/or an adapter for setting on a hollow needle, arranged at the opposite second face, in which the hollow needle fixing device is arranged offset from the center axis of the hollow needle holder in a radial direction. This arrangement has the result that the hollow needle or the adapter does not project upward in the center of the hollow needle holder, but rather is offset towards the edge of the hollow needle holder.

Since the hollow needle generally has to be set at a flat angle, for example on the arm of the patient, in order to be able to be inserted into the vein, the insertion angle is limited by the hollow needle holder that rests against the patient's arm, in the case of a hollow needle fixing device with a hollow needle, arranged in the center of the hollow needle holder. In practice, this requirement leads to the result that the hollow needle must be bent away when it is inserted into the vein of the patient, while applying force, thereby increasing the risk of injury and the difficulties in taking blood samples. If the hollow needle is arranged offset towards the edge of the hollow needle holder, according to the invention, smaller insertion angles are possible if the hollow needle holder is held appropriately, so that the hollow needle can be inserted into the arm at a flatter angle, overall. This arrangement significantly improves the handling of the hollow needle holder when the blood sample is taken. This solution according to the invention, which also constitutes an object of the present invention in itself, can be connected with the solution for disposal of the hollow needle as described above, in particularly advantageous manner, in that the unlocking mechanism interacts with the hollow needle fixing device that is arranged offset so that the hollow needle and/or the adapter falls into the hollow needle container after activation of the unlocking mechanism. In this connection, the configurations described above can be applied appropriately.

In a preferred embodiment according to the invention, the hollow needle or the adapter can be accommodated in the hollow needle fixing device so as to rotate, in order to be able to turn the ground point of the hollow needle to any desired position. This arrangement is particularly advantageous for a hollow needle holder having a hollow needle arranged in offset manner, because the hollow needle holder with the hollow needle is then no longer structured with rotation symmetry, and there is a preferred direction for the ground point of the hollow needle. This arrangement can be easily achieved using a plug-in connector. Alternatively, the correct alignment of the ground point of the hollow needle can also be predetermined by means of appropriate guidance at the hollow needle and the hollow needle fixing device.

### BRIEF DESCRIPTION OF THE DRAWINGS

Other objects and features of the present invention will become apparent from the following detailed description considered in connection with the accompanying drawings. It should be understood, however, that the drawings are designed for the purpose of illustration only and not as a definition of the limits of the invention.

In the drawings, wherein similar reference characters denote similar elements throughout the several views:

FIG. 1 is a cross-section through a first embodiment of a hollow needle holder according to the invention, with a hollow needle inserted;

FIG. 2 is a cross-section through the hollow needle holder according to FIG. 1, rotated by 90°, with a hollow needle accommodated in the hollow needle holder;

FIG. 3 is a top view of the hollow needle holder according to FIG. 1;

FIG. 4 is a top view of the second face of the hollow needle container of the hollow needle holder according to FIG. 1;

FIG. 5 is a cross-section through a hollow needle fixing device according to the invention;

FIG. 6 is a top view of-the hollow needle fixing device according to FIG. 5;

FIG. 7 is a cross-section through a second embodiment of a hollow needle holder according to the invention, with a hollow needle inserted;

FIG. 8 is a cross-section through the hollow needle holder according to FIG. 7, rotated by 90°, with a hollow needle accommodated in the hollow needle holder;

FIG. 9 a detail view of one end of the hollow needle holder in the view according to FIG. 7;

FIG. 10 is a detail view of one end of the hollow needle holder in the view according to FIG. 8;

FIG. 11 is a side view, a front view, and a top view of a slide according to the invention;

FIG. 12 is a cap according to the invention, in a view from below;

FIG. 13 is a cross-section through a cap according to FIG. 7;

FIG. 14 is a top view of a hollow needle holder according to FIG. 7;

FIG. 15 is a cross-section through a third embodiment of a hollow needle holder according to the invention, with a hollow needle inserted;

FIG. 16 is a cross-section through the hollow needle holder according to FIG. 15, rotated by 90°, with a hollow needle accommodated in the hollow needle holder;

FIG. 17 is a detail view of one end of the hollow needle holder in the view according to FIG. 15;

FIG. 18 is a cross-section through a fourth embodiment of a hollow needle holder according to the invention, with a hollow needle inserted;

FIG. 19 is a transverse cross-section through the hollow needle holder according to FIG. 18, rotated by 90°, with a hollow needle accommodated in the hollow needle holder;

FIG. 20 is a detail view of the one end of the hollow needle holder in the view according to FIG. 18;

FIG. 21 is a top view of the second face of the hollow needle holder according to FIG. 20;

FIG. 22 is a view of a slide, according to the invention, of the hollow needle holder according to FIG. 18;

FIG. 23 is a cross-section through a cap of the hollow needle holder according to FIG. 18;

FIG. 24 is a side view, a side view rotated by 90°, and a top view of a hollow needle fixing device according to FIG. 18;

FIG. 25 is a hollow needle fixing device according to the invention, according to another embodiment of the present invention;

FIG. 26 is a hollow needle according to the invention, for the hollow needle fixing device according to FIG. 25.

### DETAILED DESCRIPTION OF PREFERRED EMBODIMENTS

Turning now in detail to the drawings, FIG. 1 shows a hollow needle holder 1 having a long, cylinder-shaped hollow needle container 2, which has an opening 3, which can be closed, on the bottom face in the drawing. A hollow needle fixing device 4, mounted from the inside, is arranged on the opposite, second face of hollow needle container 2, and a commercially available hollow needle 5, which projects all the way into hollow needle container 2 through hollow needle fixing device 4, is screwed into it.

In order to take a blood sample, the free end of hollow needle 5 is inserted into the vein of the patient and a blood sample tube, not shown, is set onto the end of hollow needle 5 that is located in hollow needle container 2, through opening 3. After the blood sample has been taken, the blood sample tube is pulled off again and hollow needle 5 is withdrawn from the vein of the patient. Hollow needle holder 1, together with hollow needle 5, must now be disposed of. In order to preclude the possibility of a needle stick injury, hollow needle 5 must therefore first be introduced into hollow needle container 2.

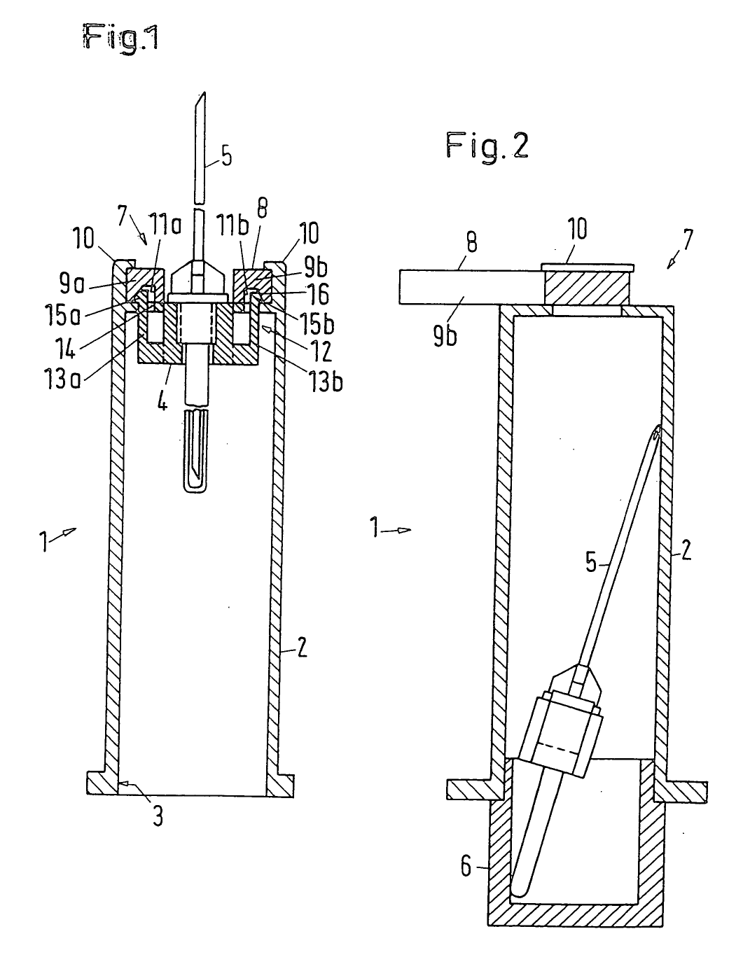

For this purpose, opening 3 is first closed with a cap 6, which is simply set on (see FIG. 2). Afterwards, an unlocking mechanism 7 is activated, which is affixed to the second face of hollow needle container 2 and is configured so that hollow needle 5 drops into hollow needle container 2 after activation of unlocking mechanism 7.

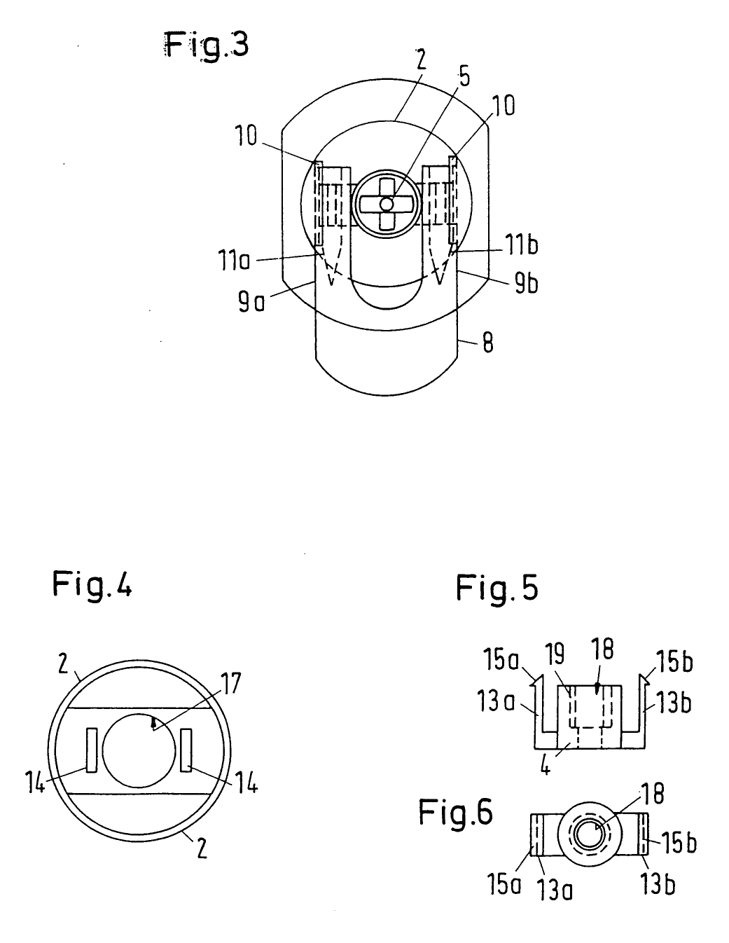

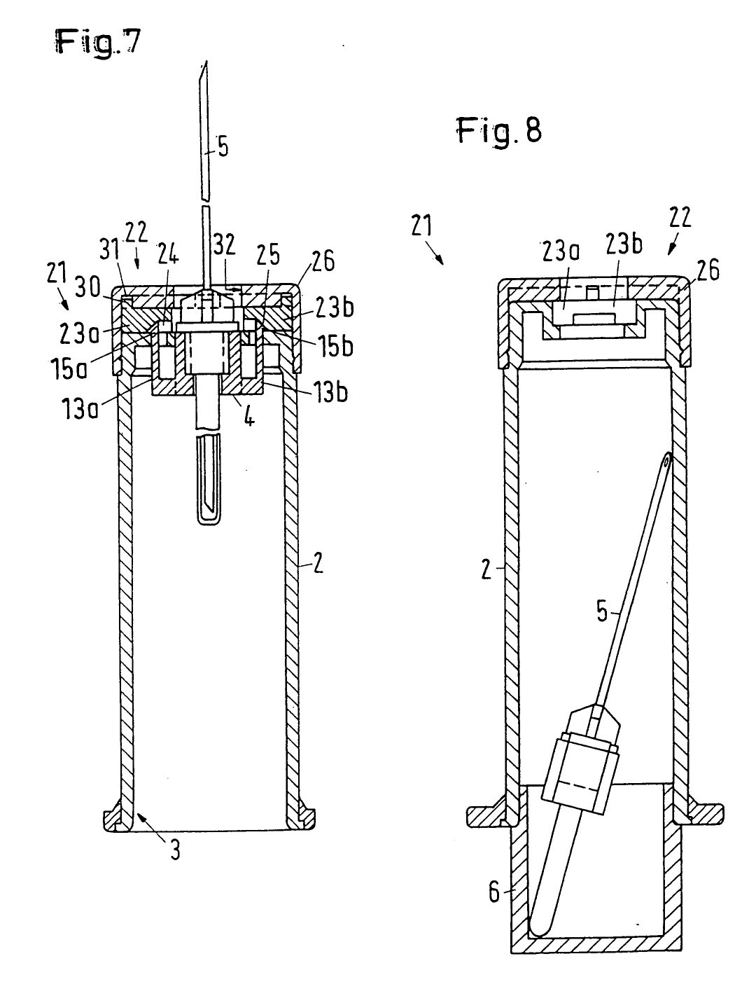

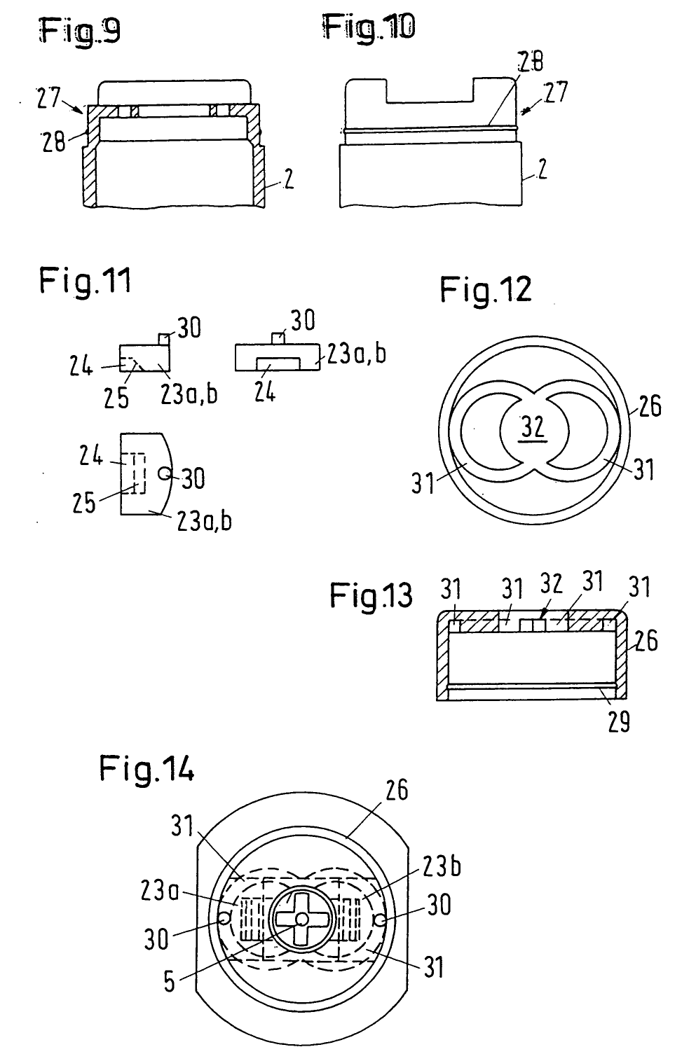

Unlocking mechanism 7 has a slide 8 having two arms 9a, 9b (see FIG. 3), which is held and guided by an angled projection 10 formed on hollow needle container 2. Projection 10 is formed on two opposite sides of hollow needle container 2. In their underside facing hollow needle container 2, arms 9a, 9b have grooves 11a, 11b that serve as guides.

These grooves 11a, 11b engage with an anchoring device 12 of hollow needle fixing device 4, which is formed by two flexible arms 13a and 13b, whereby arms 13a, 13b are configured on the side of hollow needle fixing device 4 and are each passed through an attachment opening 14 configured in the second face of hollow needle container 2. Wedge-shaped projections 15a, 15b that are directed outward, in each instance, are formed on the ends of arms 13a, 13b that project out of hollow needle container 2, which projections engage with a step on the outside of the hollow needle container that is directed laterally to the outside, and narrow in wedge shape towards their end.

Wedge-shaped projections 15a, 15b engage in grooves 11a, 11b of slide 8. In this connection, each groove 11a, 11b has a beveled side 16 that corresponds to the wedge shape of projections 15a, 15b and rests against projections 15a, 15b. Grooves 11a, 11b start at a constant width at the free end of arms 9a, 9b, and narrow towards the segment of slide 8 that connects the two arms 9a, 9b, as can be seen in FIG. 3. If the essentially horseshoe-shaped slide 8 is pressed towards the center from its end that projects over the edge of hollow needle holder 2, beveled sides 16 of narrowing grooves 11a, 11b exert a force on projections 15a, 15b, so that flexible arms 13a, 13b are pressed together inwards, until the steps of projections 15a, 15b lose their hold on the edge of attachment openings 14 and hollow needle fixing device 4 falls into hollow needle container 2, together with hollow needle 5. For this purpose, grooves 11a, 11b and attachment openings 14 are configured to be broader than projections 15a, 15b. In this connection, grooves 11a, 11b as well as the width of hollow needle fixing device 4 prevent hollow needle 5 with hollow needle fixing device 4 from falling off hollow needle holder 1.

If slide 8 is pressed further, its segment that connects arms 9a, 9b covers attachment opening 14 as well as a central passage opening 17 formed between attachment openings 14, which can be seen in FIG. 4, which represents the second face of hollow needle container 2. In this way, hollow needle holder 1 is reliably closed and any exit of the hollow needle accommodated in hollow needle container 2 is reliably precluded.

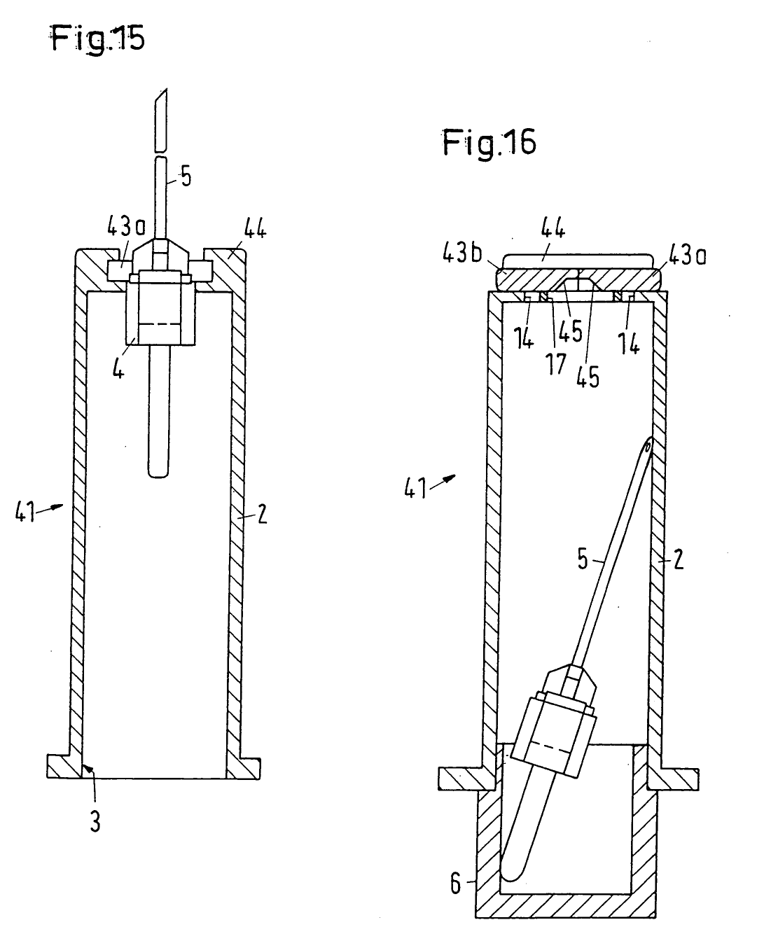

FIG. 5 shows the hollow needle fixing device 4 with flexible arms 13a, 13b in cross-section. In this connection, the central segment of hollow needle fixing device 4 has a passage 18 with an inside thread 19 that is accessible from the outside in the assembled state, into which commercially available hollow needles 5 can be screwed. The central segment of cylinder-shaped hollow needle fixing device 4 is adapted, with its outside diameter, to the inside diameter of passage opening 17, so that hollow needle fixing device 4 rests against hollow needle container 2 there and is stabilized. As is evident from FIG. 6, flexible arms 13a, 13b are connected with the hollow needle holder by way of bridges.

FIG. 7 shows a second embodiment of a hollow needle holder according to the invention. The hollow needle holder 21 is similar to hollow needle holder 1 of the first embodiment described above, in terms of its fundamental structure. In particular, hollow needle fixing device 4 and hollow needle container 2 are configured in comparable manner. Therefore no detailed description of these parts will be presented, and for the sake of simplicity, they are provided with the same reference symbols.

Hollow needle holder 21 has an unlocking mechanism 22 that differs from hollow needle holder 1, which is equipped with two slides 23a and 23b, which are configured with the same construction. As is evident from FIG. 7, slides 23a, 23b are arranged on the second face of hollow needle container 2. Hollow needle fixing device 4 is mounted on the second face from the inside of hollow needle container 2, as in the first embodiment. In this connection, each slide 23a, 23b has a recess 24 at its bottom, into which a projection 15a, 15b of flexible arms 13a, 13b of the hollow needle fixing device 4 is accommodated, in each instance. In this connection, wedge-shaped projections 15a, 15b rest against a beveled side 25 of recess 24.

Slide 23a, 23b is configured in essentially rectangular manner, whereby one side is adapted to the rounded shape of hollow needle container 2. In this way, slide 23a, 23b can rest completely on the face of hollow needle container 2, and particularly does not project beyond it. The two slides 23a, 23b are held in place by means of a cap 26 having a central passage 32 to pass the hollow needle through. Cap 26 is set onto the face of hollow needle holder 21 from the outside and surrounds slides 23a and 23b and fixes them in place. For this purpose, hollow needle container 2 is provided, on its top side edge, with a step 27 that faces towards the inside, onto which cap 26 can be set. In order to hold cap 26 in place, an elevation 28 is formed in the region of step 27 on the side edge of the housing, which elevation engages in a correspondingly formed groove 29 in cap 26. Detail views of step 27 and cap 26 are shown in FIGS. 9, 10, and 13.

In the operating position of hollow needle holder 1, with hollow needle 5 screwed on and ready for use, slide 23a, 23b is arranged between hollow needle 5 and the edge of hollow needle container 2 so that the rounded edge of slide 23a, 23b aligns in coverage with the edge of hollow needle holder 2. For activation of slide 23a, 23b, a nipple 30 that serves as a guide element is provided on its top (see FIG. 11), which is guided in a correspondingly configured groove 31 of cap 26. Groove 31 in cap 26 is configured in the shape of a double ring, as shown in FIG. 12, so that nipple 30 of each slide 23a, 23b is moved forward when cap 26 is rotated to the left or to the right, and thereby pushes slide 23a, 23b forward along with it. In this connection, beveled edge 25 of slide 23a, 23b pushes projections 15a, 15b of hollow needle fixing device 4 out of the anchoring device, so that hollow needle 5, with the hollow needle fixing device, falls into hollow needle container 2 (see FIG. 8). After cap 26 is turned further by 90°, slides 23a and 23b that are arranged opposite one another and suitably guided on the face of hollow needle container 2 come together in the middle and close passage opening 17 and attachment openings 14. FIG. 14 shows unlocking mechanism 22 in a top view.

FIG. 15 shows a third embodiment of a hollow needle holder 41 according to the invention, which is similar, in its fundamental structure, to hollow needle holder 1 of the first embodiment described above. Hollow needle container 2, and hollow needle fixing device 4, in particular, are configured in comparable manner. Therefore no detailed description of these parts will be presented, and for the sake of simplicity, they are provided with the same reference symbols.

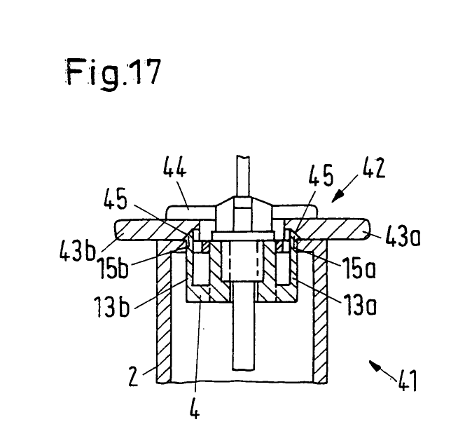

Hollow needle holder 41 has an unlocking mechanism 42 that is different from hollow needle holders 1 and 21, which can be seen in detail in FIG. 17. Unlocking mechanism 42 contains two slides 43a, 43b having the same construction, which are arranged on opposite sides of hollow needle fixing device 4 and are held and guided by means of projections 44 molded onto hollow needle container 2. When hollow needle fixing device 4 is inserted, beveled sides 45 of slides 43a, 43b rest against projections 15a, 15b that form the anchoring device of hollow needle fixing device 4.

In order to release hollow needle fixing device 4 from hollow needle holder 41, the two slides 43a, 43b are pressed together towards the inside, towards one another. In this way, flexible arms 13a, 13b of hollow needle fixing device 4 are also pressed far enough together so that the anchoring of projections 15a, 15b is released and hollow needle fixing device 4, with hollow needle 5, falls into hollow needle container 2 that is closed by means of a cap 6. By pressing slides 43a, 43b together further, until they come together in the center of hollow needle holder 41, attachment openings 14 and passage opening 17 in the second face of hollow needle container 2 are furthermore reliably closed (see FIG. 16). In comparison with one slide 8 of hollow needle holder 1 according to the first embodiment, the two slides 43a, 43b are fixed in place on the hollow needle holder 41 rotated by 90°, and project less beyond the edge of the hollow needle container 2.

A directly acting and simple unlocking mechanism 42 is achieved by means of the structure described above. This arrangement facilitates handling in practice.

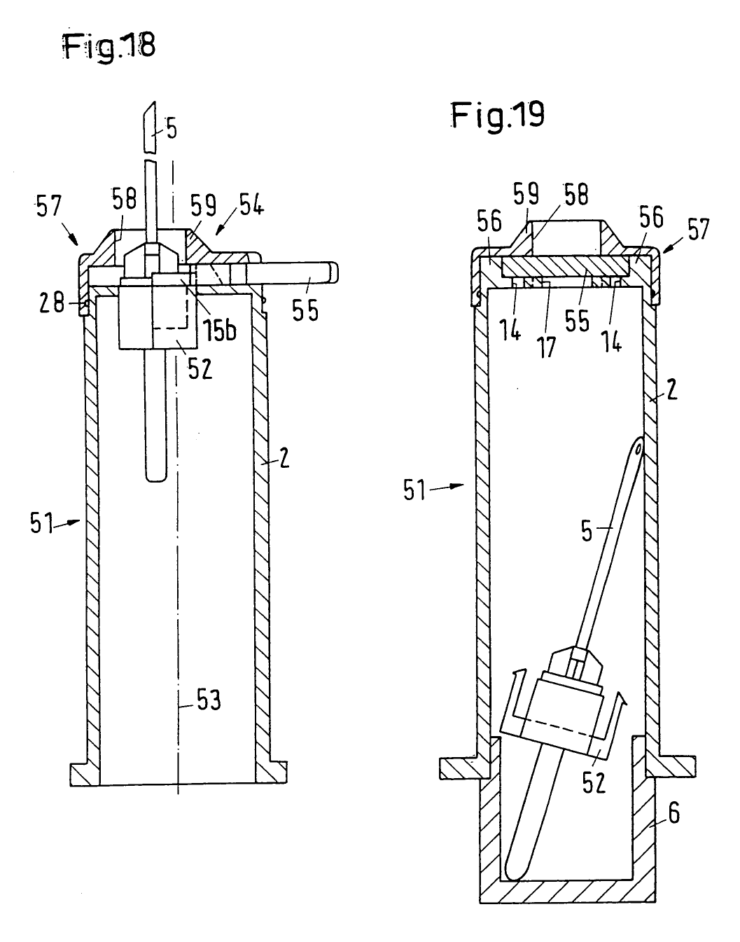

A fourth embodiment of a hollow needle holder 51 according to the invention is shown in FIG. 18. Again, the same parts, relative to the previous embodiments, are provided with identical reference symbols, and will not be described in detail.

The essential difference between hollow needle holder 51 and the embodiments described above lies in the arrangement and the structure of hollow needle fixing device 52. As can be seen in FIG. 18, hollow needle fixing device 52 and hollow needle 5 accommodated in it are arranged offset from center axis 53 of hollow needle holder 51 in a radial direction. As a result, hollow needle 5 is located closer to one edge of hollow needle holder 51 and can be inserted into the vein of a patient more easily.

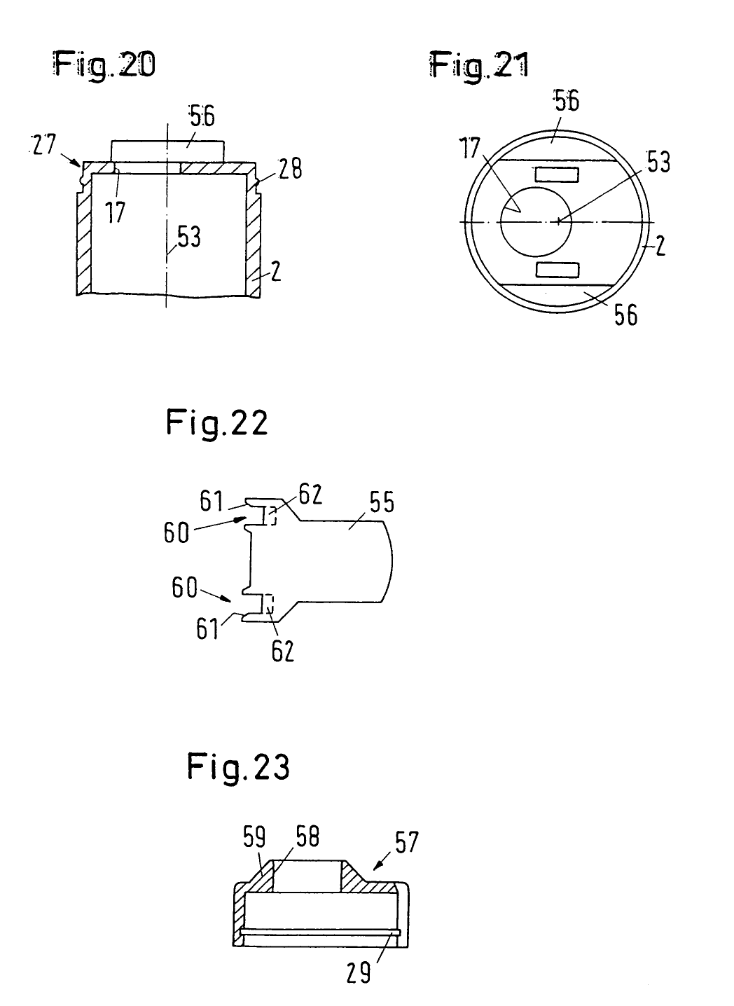

As is evident from FIG. 21, passage opening 17 is arranged in the face of hollow needle container 2 offset relative to center axis 53, for this purpose. Attachment openings 14, on the other hand, are distributed symmetrically about the center axis, in their longitudinal direction. On the basis of this arrangement, hollow needle 5 that projects through passage opening 17 is also offset towards the edge of hollow needle holder 51. The offset between center axis 53 and the center of passage opening 17 is also clearly evident in FIG. 20.

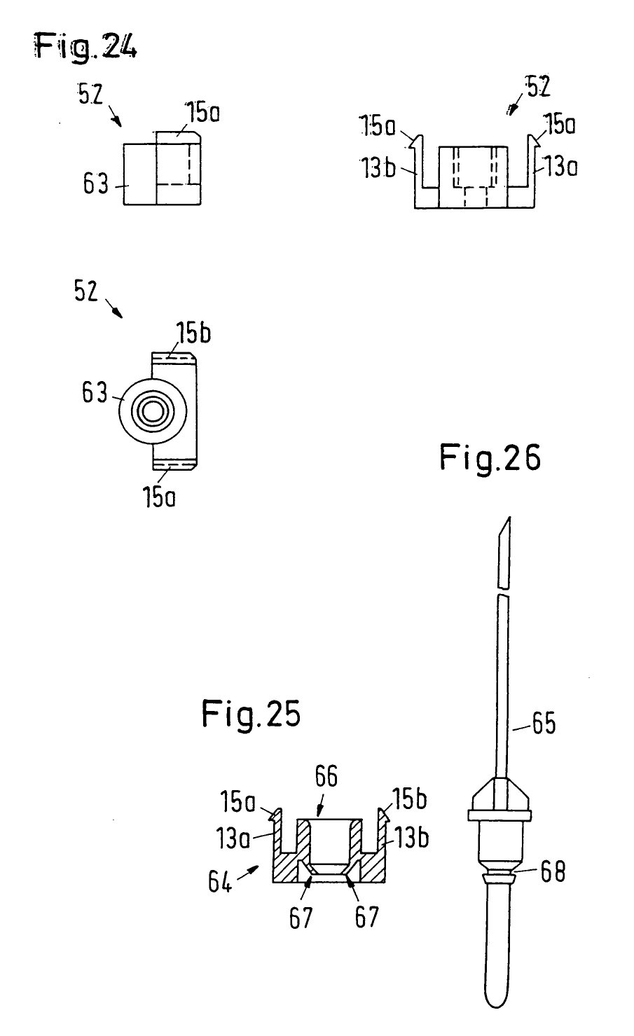

In accordance with the previous embodiments, an unlocking mechanism 54 having a slide 55 is provided, which engages at projections 15a, 15b of the anchoring device of hollow needle fixing device 52 by means of its corresponding shape and presses projections 15a, 15b together, together with flexible arms 13a, 13b (see FIG. 24), when it is pushed inwards. As a result, hollow needle fixing device 52 is released from hollow needle holder 51, and hollow needle 5 falls into hollow needle container 2, together with hollow needle fixing device 52, as shown in FIG. 19. By pushing slide 55 further, passage opening 17 and attachment openings 14 are closed.

The slide is guided laterally by means of projections 56 on hollow needle container 2 (see FIGS. 20 and 21) and held, towards the top, by a cap 57, which has an opening 58 having an edge reinforcement 59 at the location corresponding to passage opening 17. Cap 57 is fixed in place on hollow needle container 2 by means of setting it on, similar to cap 26 according to the second embodiment.

Slide 55 shown in detail in FIG. 22 is structured in fork shape and has two recesses 60 that engage at projections 15a, 15b of hollow needle fixing device 52. For this purpose, beveled sides 61, which press projections 15a, 15b together, and beveled surfaces 62 are provided on the recesses, in each instance, which press projections 15a, 15b downward in the direction of hollow needle container 2.

As the detail representation of FIG. 24 shows, a holder 63 that serves for the direct accommodation of hollow needle 5 is affixed, offset relative to flexible arms 13a, 13b having the projections, i.e. anchoring devices 15a, 15b, in order to be arranged in accordance with passage opening 17 in the assembled state.

FIG. 25 shows a particular embodiment of a hollow needle fixing device 64, into which a hollow needle 65 can be engaged by means of a plug-in connector 66. For this purpose, hollow needle fixing device 64 has projections 67 that engage in a corresponding groove 68 of hollow needle 65. This arrangement has the advantage that hollow needle 65 can be rotated in hollow needle fixing device 64. This feature is particularly advantageous in the case of the hollow needle fixing device that is arranged with an offset, according to the fourth embodiment, in order to align the ground point of the hollow needle in optimal manner. Hollow needle fixing device 64 can, however, be configured to be compatible with all the hollow needle fixing devices of the embodiments described above.

Using hollow needle holder 1, 21, 41, and 51 according to the invention, a particularly safe, quick, and simple disposal of the hollow needle 5, 65 as well as simple handling are guaranteed. Since hollow needle 5, 65 falls into holder 1, 21, 41, 51, i.e. into its hollow needle container 2, the risk of injury due to inaccurate ejection is excluded, and handling is therefore decisively improved for the personnel.

Although several embodiments of the present invention have been shown and described, it is to be understood that many changes and modifications may be made thereunto without departing from the spirit and scope of the invention as defined in the appended claims.

1. A hollow needle holder comprising:
(a) a hollow needle container having a first face comprising a closable opening and a second face;
(b) a hollow needle fixing device disposed on said second face for receipt of a hollow needle or an adapter for a hollow needle projecting through said hollow needle fixing device into said hollow needle container; and
(c) an unlocking mechanism for releasing the hollow needle or adapter after use so that the hollow needle or adapter falls into said hollow needle container after activation of said unlocking mechanism.

2. The hollow needle holder according to claim 1, further comprising an anchoring device releasably fixing said hollow needle fixing device in place on said hollow needle holder.

3. The hollow needle holder according to claim 1, wherein said hollow needle fixing device is engageable on the hollow needle holder.

4. The hollow needle holder according to claim 2, wherein the hollow needle holder has a passage opening for the hollow needle or the adapter and at least one attachment opening for said hollow needle fixing device on said second face.

5. The hollow needle holder according to claim 1, wherein said hollow needle fixing device has at least one flexible arm having a wedge-shaped projection for engagement on said hollow needle holder.

6. The hollow needle holder according to claim 2, wherein said unlocking mechanism has at least one slide for releasing said anchoring device.

7. The hollow needle holder according to claim 6, wherein said at least one slide has a guide engaging said anchoring device.

8. The hollow needle holder according to claim 6, wherein two slides are provided.

9. The hollow needle holder according to claim 6, wherein said at least one slide projects beyond an edge of said hollow needle container.

10. The hollow needle holder according to claim 6, wherein said unlocking mechanism has a cap arranged to rotate on said hollow needle container, said at least one slide being arranged so that rotation of said cap moves said at least one slide.

11. The hollow needle holder according to claim 10, wherein said cap has a guide and said at least one slide has a guide element engaging said guide.

12. The hollow needle holder according to claim 1, wherein said unlocking mechanism has a closure device for closing said hollow needle container.

13. The hollow needle holder according to claim 12, wherein said closure device comprises at least one slide.

14. The hollow needle holder according to claim 1, wherein said hollow needle fixing device is arranged offset from a center axis of said hollow needle holder in a radial direction.

15. The hollow needle holder according to claim 1, further comprising a plug-in connector disposed in said hollow needle fixing devise for rotatably mounting said hollow needle or adapter.
